(19) United States
(12) Patent Application Publication
(10) Pub. No.: US 2026/0298823 A1
(43) Pub. Date: Oct. 1, 2026

(54) DETECTION OF EXPLOSIVE SAMPLES

(71) Applicant: ALTI LLC, Highland Park, IL (US)

(72) Inventor: James McChesney Hargrove, Highland Park, IL (US)

(21) Appl. No.: 19/091,359

(22) Filed: Mar. 26, 2025

(51) Int. Cl. G01N 21/64; G01N 1/22; G01N 1/44; G01N 33/00

(52) U.S. Cl. CPC G01N 21/6402; G01N 1/2273; G01N 1/44; G01N 21/645; G01N 33/0057

(57) ABSTRACT

A method of sensing explosives can include sampling an atmosphere over a sample; and detecting explosive impurities or decomposition products in the atmosphere. The detecting may be performed by cavity ring-down spectroscopy or laser induced fluorescence. The explosives may be at least one of cyclotrimethylene trinitramine (RDX), 1,3,5,7-Tetranitro-1,3,5,7-tetrazocane (HMX), TNT, PETN, nitroglycerin, triaminotrinitrobenzene, triacetonetriperoxide or sodium nitrate.

### TECHNICAL FIELD

Embodiments are generally related to a device and method to detect the trace odor of explosives by sensing the vapor partial pressure of the explosives and/or their decomposition products and impurities.

### BACKGROUND

The transport of explosives samples is heavily regulated, for example, by 37 C.F.R. § 173. Explosives forbidden for transportation include unapproved explosives, chlorate-containing explosives, a leaking or damaged package containing explosives, unstable or deteriorated propellants, nitroglycerin, diethylene glycol dinitrate, or any other liquid explosives, a loaded firearm, fireworks that combine an explosive or a detonator, fireworks containing yellow or white phosphorous, large toy torpedoes, or an explosive article with its means of initiation or ignition installed.

Explosives, including new explosives, are assigned a class and division as follows: Division 1.1 if the major hazard is mass explosion; Division 1.2 if the major hazard is dangerous projections; Division 1.3 if the major hazard is radiant heat or violent burning, or both, but there is no blast or projection hazard; Division 1.4 if there is a small hazard with no mass explosion and no projection of fragments of appreciable size or range; Division 1.4 Compatibility Group S (1.4S) if the hazardous effects are confined within the package or the blast and projection effects do not significantly hinder emergency response efforts. The determination whether or not inclusion in the explosive class is proper depends on if the substance or article does not have significant explosive hazard or if the effects of explosion are completely confined within the article.

The Occupational Safety and Health Administration sets forth the following classes of explosives: Class A explosives-Possessing, detonating, or otherwise maximum hazard; such as dynamite, nitroglycerin, picric acid, lead azide, fulminate of mercury, black powder, blasting caps, and detonating primers. Class B explosives-Possessing flammable hazard, such as propellant explosives (including some smokeless propellants), photographic flash powders, and some special fireworks. Class C explosives-Includes certain types of manufactured articles which contain Class A or Class B explosives, or both, as components but in restricted quantities. Forbidden or not acceptable explosives-Explosives which are forbidden or not acceptable for transportation by common carriers by rail freight, rail express, highway, or water in accordance with the regulations of the U.S. Department of Transportation, 49 C.F.R. chapter I.

In order to determine the presence of explosives, testing is necessary. For example, TSA checkpoints at airports utilize Explosives Trace Detection (ETD) tests. The EDT test uses a swab on such things as laptops, shoes, film, cell phones, bags, wheelchairs, hands, casts, etc. The ETD must detect and identify the explosives as particles or residues at the trace levels specified in the sensitive criteria when employed in the operational environment by representative personnel. The sensitive criteria identify the types and quantities of explosive materials (explosive trace) that must be detected, the minimum detection rate for each category of explosive, and the overall detection and maximum nuisance alarm rates. The criteria also specify the requirement to detect the minimum quantity and larger quantities of each listed explosive.

There are conventional methods of detecting trace explosive vapors, including mass spectroscopy, fluorescence methods, chemosensors, chromatography, photoacoustic spectroscopy, or enzyme linked immunoassays (ELISAs). However, these methods frequently entail expensive equipment, have high maintenance costs, require highly trained personnel and may be time consuming to perform. For example, fluorescence technology may require changing a fluorescent card monthly or even more frequently.

Moreover, a complicating factor is the very low volatility of some classes of explosives, for example cyclonite (RDX), which is 1,3,5-Trinitroperhydro-1,3,5-triazine. RDX is often used in mixtures with other explosives and plasticizers or phlegmatizers (desensitizers); it is the explosive agent in C-4 plastic explosive and a key ingredient in Semtex. The situation is similar for octogen (HMX) which stands for High Melting explosive, and is cyclotetramethylene-tetranitramine. The low volatility of these compounds often hinder quick and easy detection by conventional detection methods, but both HMX and RDX are expected to produce degradation products that are more volatile.

There is accordingly a need for new and improved methods to detect explosive samples.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

The disclosure, in part, pertains to a method of sensing explosives, which includes sampling an atmosphere over a sample; and detecting explosive impurities or decomposition products in the atmosphere. The detecting can be performed by thermal decomposition cavity ring-down spectroscopy, by thermal desorption thermal decomposition cavity ring-down spectroscopy, by thermal decomposition laser induced fluorescence, or by thermal desorption thermal decomposition laser induced fluorescence. The explosives can be at least one of cyclotrimethylene trinitramine, 1,3,5,7-Tetranitro-1,3,5,7-tetrazocane, TNT, PETN, nitroglycerin, triaminotrinitrobenzene, triacetonetriperoxide, potassium nitrate or sodium nitrate.

In the disclosure, the explosive may be cyclotrimethylene trinitramine, ammonium perchlorate, ammonium picrate, 1,2-bis-(2,2-difluoro-2-nitroacetoxyethane), black powder, bis (trinitroethyl) carbonate, bis (trinitroethyl) nitramine, 1,2,4-butanetriol trinitrate, calcium nitrate explosive mixture, cellulose hexanitrate explosive mixture, chlorate explosive mixtures, cyclotetramethylenetetranitramine, diaminotrinitrobenzene, diazodinitrophenol, diethyleneglycol dinitrate, dimethylol, dinitroethyleneurea, dinitroglycerine, dinitrophenol, dinitrophenolates, dinitrophenyl hydrazine, dinitroresorcinol, dinitrotoluene-sodium nitrate explosive mixtures, dipicramide; diaminohexanitrobiphenyl, dipicryl sulfide, dipicryl sulfone, dipicrylamine, 2,2-dinitropropyl acrylate, dinitropentano nitrile, ethylene diamine dinitrate, ethylenedinitramine, ednatol, ethyl 4,4-dinitropentanoate, ethylene glycol dinitrate, erythritol tetranitrate explosives, esters of nitro-substituted alcohols, ethyl-tetryl, tetranitromethane (nitroform), fulminate of mercury, fulminate of silver, gelatinized nitrocellulose, guanyl nitrosamino guanyl tetrazene, guanyl nitrosamino guanylidene hydrazine, guncotton, hexanitrodiphenylamine, hexanitrostilbene, hexamethylenetriperoxidediamine, cyclo-1,3,5,7-tetramethylene, 2,4,6,8-tetranitramine, octogen, potassium dinitrobenzo-furoxane, lead azide, lead mannite, lead mononitroresorcinate, lead picrate, mannitol hexanitrate, methyl 4,4-dinitropentanoate, monoethanolamine nitrate, mercuric fulminate, mercury oxalate, mercury tartrate, metriol trinitrate, monomethylamine nitrate; methylamine nitrate, mononitrotoluene-nitroglycerin mixture, nitroisobutametriol trinitrate, nitrate sensitized with gelled nitroparaffin, nitrated carbohydrate explosive, nitrated glucoside explosive, nitrated polyhydric alcohol explosives, nitric acid and a nitro aromatic compound explosive, nitric acid and carboxylic fuel explosive, nitric acid explosive mixtures, nitro aromatic explosive mixtures, nitro compounds of furane explosive mixtures, nitrocellulose explosive, nitroderivative of urea explosive mixture, nitrogelatin explosive, nitrogen trichloride, nitrogen tri-iodide, nitroglycerine, nitroglycide, nitroglycol, nitroguanidine explosives, nitronium perchlorate propellant mixtures, nitroparaffins explosive grade and ammonium nitrate mixtures, nitrostarch, nitro-substituted carboxylic acids, 3-nitro-1,2,4-triazol-5-one, nitrourea, silver acetylide, silver azide, silver fulminate, silver oxalate explosive mixtures, silver styphnate, silver tartrate explosive mixtures, silver tetrazene, sodium azide explosive mixture, sodium dinitro-ortho-cresolate, sodium nitrate explosive mixtures, sodium nitrate-potassium nitrate explosive mixture, sodium picramate, tetranitro-2,3,5,6-dibenzo-1,3a,4,6a-tetrazapentalene, triaminotrinitrobenzene, triacetonetriperoxide, triethylene glycol dinitrate, tetranitrocarbazole, tetrazene tetracene, tetrazine, 1 (5-tetrazolyl)-4-guanyl tetrazene hydrate, tetrazole explosives, tetryl 2,4,6-tetranitro-n-methylaniline, tetrytol, thickened inorganic oxidizer salt slurried explosive mixture, trimethylolethane trinitrate, trinitroethyl formal, trimethylol ethyl methane trinitrate composition, trimethylolthane trinitrate-nitrocellulose, trimonite, trinitroanisole, trinitrobenzene, trinitrobenzenesulfonic acid picryl sulfonic acid, trinitrobenzoic acid, trinitrocresol, trinitrofluorenone, trinitro-meta-cresol, trinitronaphthalene, trinitrophenetol, trinitrophloroglucinol, trinitroresorcinol, tritonal or urea nitrate.

In the disclosure, the detecting may be at parts per billion and parts per trillion levels, and cyclotrimethylene trinitramine and 1,3,5,7-Tetranitro-1,3,5,7-tetrazocane are detected at parts per billion or parts per trillion levels when their vapor partial pressures are 10,000 and 1,000,000 times lower.

The disclosure, in part, pertains to an apparatus that performs the method of the disclosure, which includes an inlet; a particulate filter; and a detector. The apparatus can further include a thermoelectric element configured to heat or cool the sample and an optional capillary column before the inlet, and a NO generator configured to feed NO into the atmosphere prior to the detector. The detector can be a thermal decomposition cavity ring-down spectrometer, a thermal desorption thermal decomposition cavity ring-down spectrometer, a thermal decomposition laser induced fluorescence detector, or a thermal desorption thermal decomposition laser induced fluorescence detector.

The disclosure, in part, pertains to a method of sensing explosives that includes sampling an atmosphere over a sample; and detecting explosive impurities or decomposition products in the atmosphere, where the explosives include at least one of cyclotrimethylene trinitramine, 1,3,5,7-Tetranitro-1,3,5,7-tetrazocane, TNT, PETN, nitroglycerin, triaminotrinitrobenzene, triacetonetriperoxide or sodium nitrate. The detector can be a thermal decomposition cavity ring-down spectrometer, a thermal desorption thermal decomposition cavity ring-down spectrometer, a thermal decomposition laser induced fluorescence detector, or a thermal desorption thermal decomposition laser induced fluorescence detector. In the method, cyclotrimethylene trinitramine and 1,3,5,7-Tetranitro-1,3,5,7-tetrazocane are detected at parts per billion levels when their vapor pressures are 10,000 and 1,000,000 times lower. An apparatus performing the method can include an inlet; a particulate filter; and a detector. A thermoelectric element configured to heat or cool the sample and an optional capillary column can be before the inlet. An NO generator can be configured to feed NO into the atmosphere prior to the detector. The detector may be a thermal desorption thermal decomposition cavity ring-down spectrometer, a thermal decomposition laser induced fluorescence detector, or a thermal desorption thermal decomposition laser induced fluorescence detector.

### BRIEF DESCRIPTION OF THE DRAWINGS

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1 shows a block diagram of a Cavity Ring Down Spectrometer (CRDS) analyzer according to embodiments of the disclosure

FIG. 2 is a simplified diagram of a laser induced fluorescence (LIF) spectrometer according to an embodiment of the disclosure.

FIG. 3 shows the threshold level for a TD-CRDS measurement of a sample according to an embodiment of the disclosure.

FIG. 4 shows the level of sensitivity as achievable for a wide range of explosive materials according to an embodiment of the disclosure.

### DETAILED DESCRIPTION

The particular values and configurations discussed in the following non-limiting examples can be varied and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB. AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

The technology of the present disclosure is based upon the vapor partial pressure of explosives and the vapor partial pressure of the decomposition products that explosives exude during storage. Additionally, explosives are not 100% pure, and the vapor partial pressure profile of the impurities can be used to enable the identification of specific explosives. Moreover, the detection of degradation products could also be used to monitor explosives and estimate the amount of aging. The detection of decomposition products from explosives could also be used to monitor explosives in storage to determine the degree of decomposition and to ensure that the explosives are still effective, and to provide an indication of when they should be replaced.

Explosives detection is an important activity undertaken for military, counter-terrorism and homeland security applications. The vapor pressures (VPs) of explosive materials vary over double digit orders of magnitude, ranging from the highly volatile (e.g., diacetone diperoxide and ethylene glycol dinitrate) to the near-involatile inorganic salts (e.g., potassium chlorate). Detection of compounds with this extensive range of volatilities presents a challenge, and, historically, there have been two main approaches: direct vapor detection and particle-based detection. Direct vapor detection is difficult, at best, for all but the most volatile explosives, due to attenuation of the available vapor due to packaging, adhesion to surfaces, and diffusion in the environment. However, the technology of the disclosure opens the way to detect the presence of low volatility explosives solely by detecting the vapor partial pressure of the decomposition products after thermal conversion to NO₂.

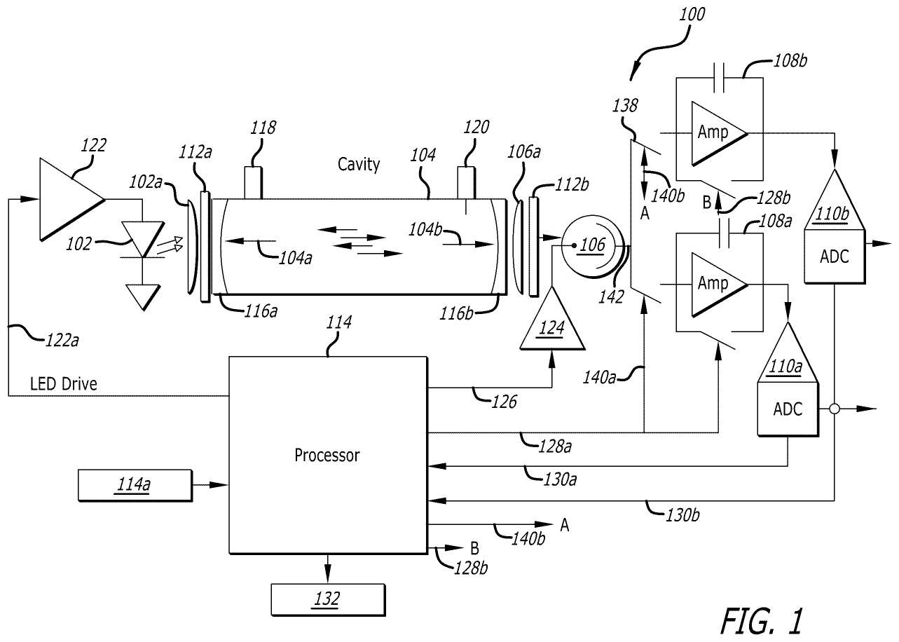

This technology is rendered viable due to the high sensitivity of spectral analyzers. An example is thermal decomposition cavity ring-down spectroscopy, TD-CRDS or thermal desorption thermal decomposition cavity ring-down spectroscopy, TD-TD-CRDS according to an embodiment shown in the block diagram of FIG. 1.

As shown in FIG. 1 in block diagram format, principle components of CRDS 100 generally include a light source 102, an optical cavity 104, a detector 106, a first integrator 108a, a second integrator 108b and a converter 110. Light source 102 with the aid of an associated lens or lenses 102a may be directed toward a proximal end 104a of optical cavity 104 while a distal end 104b of optical cavity 104 may be directed toward detector 106 with the aid of an associated lens 106a. In one embodiment, a bandpass filter 112a is positioned between light source 102 and proximal end 104a of optical cavity 104, and/or a bandpass filter 112a is positioned between distal end 104b of optical cavity 104 and detector 106 and/or proximate to the detector 112b. Detector 106 is in gated electrical communication through the switching circuitry 138, e.g., one or more field effect transistors, with integrators 108a and 108b. The integrators 108a and 108b are in electrical communication with the respective converter 110a and 110b. All components (i.e., light source 102, optical cavity 104, detector 106, integrators 108a and 108b, and converters 110a and 110b) are in electronic communication with and controlled and/or driven by a processing or controlling component, generally referred to in the figure as computer 114. An oscillator 114a may be utilized to provide a stable and accurate timing source for computer 114 instruction stepping and timing and, in turn, for the gated integration measurement intervals according to aspects disclosed herein.

The light escaping from distal end 104b of optical cavity 104 is focused on to detector 106 with the aid of lens 106a which in turn converts the photons from the light into electrons. Detector 106 collects the photons emitted from optical cavity 104 only when gated (i.e., driven ON by an amplified buffer 124). During a single ON-OFF event, portions of two separate measurements made. During a portion of the ON event, the output from the detector is directed, e.g., gated or switched into electrical communication 138 with a first integration circuit 108a. During this time the current 142 flowing from the detector 106 is integrated by integrator 108a. Immediately after the ON event the light source 102 is switched off by processor 114 to produce an OFF event. During a portion of the OFF event, the output of the detector 106 is directed through the gating circuit 138 to the second integrator 108b. The current 142 flowing from the detector 106 is then integrated by integrator 108b. A number of ON-OFF events are conducted during a single integration period of time. The sum of each of the currents from each of the portions of an ON event obtained during the integration time are integrated to pro-duce an electronic signal which represents a total ring up time. The sum of each of the currents from each of the portions of an OFF event, obtained during this same integration time, are integrated to produce an electronic signal which represents a total ring down time. The gating circuit 138 is controlled by the processor 114 via communication links 140a and 140b. The sample time signal output (arrow 126) from computer 114 to detector 106 defines this gated detection time. Each ON event and OFF event produces a 55 small current sample which is collected by integrators 108a and 108b. This process is repeated over, for example, ten-thousand to one hundred thousand times during a single integration period of time, (e.g., 10-100 μs ON-OFF cycle time over an integration period of time of about 0.1 to 1 second) which in turn creates a significant output voltage at integrators 108a and 108b. The output voltage is then measured by a corresponding converter 110a or 110b, each of which may be, for example, a high-resolution analog-to-digital (ADC) converter. After the end of the previous measurement 65 cycle (integration period of time) and before the beginning of the next measurement cycle, the integrators 108a and 108b and the corresponding circuitry may be reset (arrows 128a and 128b) by computer 114 and the initial output voltage of the integrators 108a and 108b may be measured by converters 110a and 110b, respectively, e.g., the initial output voltage of integrator 108a and 108b is measured between cycles. Measuring the initial output voltage of the integrator is more accurate than assuming the reset output voltage is “zero”.

In one or more embodiments, the cavity ring down measurement system further includes three pairs of MOSFETS, each having an N-channel and a P-channel. The first channel is to zero the photodetector, the second and the third channels are to collect alternating signals. That is, a first dual N-channel and P-channel MOSFET pair is connected to the optical detector for receiving a first detection signal; a first operational amplifier having a positive input for receiving a first dual N-channel and P-channel MOSFET pair output signal and a negative input for receiving a first operational output signal and a second dual N-channel and P-channel MOSFET pair electrically connected to the first operational output signal; and wherein the second integrating circuit comprises: a third dual N-channel and P-channel MOSFET pair connected to the detector for receiving a second signal from the detector; a second operational amplifier having an input for receiving a third dual N-channel and P-channel MOSFET pair output signal and a second input for receiving a second operational output signal.

The simultaneous gated analysis further provides a factor of improvement of 2, along with a factor of 1.9 for the path-length and 1.5 intensity resulting in a factor of 2.3 improvement. Furthermore, in embodiments the device utilizes an analysis method using a laser having a power output suitable to produce a factor of 4 improvement from 0.5 PPB of NO₂ to 30 PPT of NO₂, which for TNT results in a detection limit below 10 ppt. Reduction of systematic error may reduce that to near 3 PPT/2 seconds for NO₂, the photon limit with 25× improved mirrors which can produce a factor of 5 improvement.

Low volatility explosives can also be measured using Thermal Decomposition Laser Induced Fluorescence (LIF) or (TDLIF). LIF is an optical spectroscopic technique where a sample is excited with a laser, and the fluorescence emitted by the sample is subsequently captured by a photodetector. In LIF Electrons in the aromatic molecules will absorb light energy at a given wavelength and re-emit it in the form of fluorescence at a lower energy level. The spectra created by these emitting molecules are unique to each. An advantage of LIF over absorption spectroscopy is that it is possible to get two- and three-dimensional images since fluorescence takes place in all directions (i.e. the fluorescence signal is usually isotropic). The signal-to-noise ratio of the fluorescence signal is very high, providing a good sensitivity to the process. It is also possible to distinguish between more species, since the lasing wavelength can be tuned to a particular excitation of a given species which is not shared by other species.

LIF can be used to detect NOx, specifically NO and NO₂. LIF can directly detect NO by tuning a laser to a specific wavelength that excites NO molecules, causing them to fluoresce. While LIF can't directly detect NO₂, it can be detected indirectly by converting NO₂ to NO using a photolytic or catalytic inlet converter, and then measuring the resulting NO using LIF. Some instruments can also be used to detect other species that can be converted into NO. Since most explosives contain nitrogen, the nitrogen in the degradation products can be oxidized to NOx to permit detection by LIF. Advantages of LIF include high sensitivity to detect very low concentrations of NOx, fast response for real-time monitoring of NOx concentrations, and precise measurements allowing detection at PPT levels.

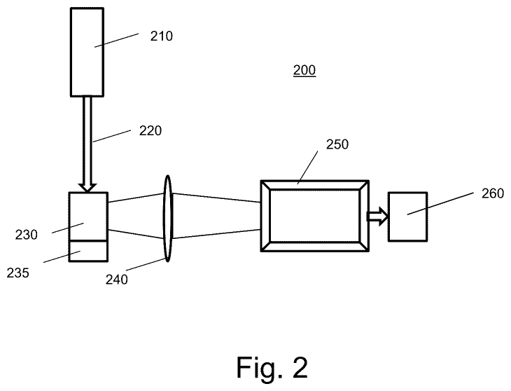

FIG. 2 is a simplified diagram of an LIF spectrometer 200. A fixed wavelength laser 210 emits a beam of monochromatic light 220 on a sample 230. For thermal decomposition, LIF (or TD-LIF), the sample may be heated with a heater 235. The fluorescence is collected by a convex mirror 240 and sent to a monochromator 250 which screens to wavelengths of the florescence to which are detected by the detector 260. Some of the available laser sources are Nd: YAG laser, dye lasers, excimer lasers, and ion laser, but for a portable system, a diode laser or quantum cascade laser may be preferred. In planar LIF. I Planar LIF the light from the laser (usually a beam) is passed through a set of lenses and/or mirrors to form a sheet, which is then used to illuminate the medium. The signal is captured by a PMT, a CCD or CMOS camera (intensified cameras can also be used). Timing electronics are often used to synchronize pulsed light sources with intensified cameras. In the disclosure, desorption of breakdown products emanates from the sample. In this aspect no heating or mild heating may be utilized. If both desorption and thermal decomposition is used, the method is referred to as thermal desorption thermal decomposition LIF (or TD-TD-LIF). Separation of the different products emanating from the explosive can be achieved by several methods. Heating at different temperatures can separate with the more volatile components being removed from the explosive before the less volatile components. Using a capillary column inline with thermal desorption could make the separation better. A thermoelectric element can both chill and heat a small sample by reversing the current by the Peltier effect. One could simply push a button to thermally reverse to heating and sample the capillary tube. This can work in a one or two channel detector, with two channels being better for differential detection. Other separation methods can be utilized, such as gas chromatography (GC) or high pressure liquid chromatography (HPLC).

An alternative measurement method would be to determine the amount of explosives in the atmosphere around the sample by thermal decomposition and chemiluminescence of NO. Chemiluminescence detectors take advantage of nitric oxide (NO) and nitrogen dioxide (NO₂) chemical reactions that emit light as part of that process. This is different from fluorescence or phosphorescence, in that the light produced stems from a chemical reaction rather than by the absorption of photons by the molecule. Chemiluminescence analyzers use a thermally stabilized photodiode to measure the intensity of the light produced by the reaction of NO with ozone (O₃). The intensity is directly proportional to the concentration of NO that was converted to NO₂ by the reaction. By converting the NO₂ in the gas stream to NO, then reacting it with the O₃, the total NOx value can be calculated, allowing speciation of NO, NO₂ and total NOx with a single analyzer.

For example, when nitrogen monoxide (NO) reacts with ozone (O₃) and is oxidized, NO changes to nitrogen dioxide (NO₂). The resulting NO₂ promotes to an excited state (NO₂) at a fixed rate and emits at a specific wavelength when it returns to the ground state of NO₂, as is shown in the following equations:

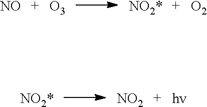

where hv is the emission (chemiluminescence) energy. This principle is used for continuous concentration measurement of nitrogen oxides (NOₓ:NO+NO₂), NO, NO₂ and ammonia (NH₃) in sample gases. The thermal decomposition of explosives produce this gases (especially NO), which can then be detected using the technology of the disclosure.

If chemiluminescense is utilized a basic pH scrubber can be used to remove NO₂ while passing the compounds of interest. The scrubber can be a quarter inch diameter piece of PFA tubing with 1-3 cm length half full of Actinite (CaOH₂ on a substrate).

The methodology can be used to test for a number of explosives. These explosives include RDX (cyclotrimethylene trinitramine—C₃H₆N₆O₆), which has the lowest vapor pressure MP=205.5° C., BP=234° C. RDX has the following chemical structure:

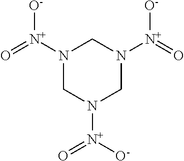 [SMILES: O=[N+]([O-])N1CN([N+](=O)[O-])CN([N+](=O)[O-])C1]

RDX, when degrading, forms various products including methylenedinitramine (MEDINA), and potentially 4-nitro-2,4-diazabutanal (NDAB) depending on the conditions, along with the nitroso-derivatives MNX, DNX, and TNX. Under anaerobic conditions, RDX degrades through nitro-reduction, forming the nitroso-derivatives: MNX (hexahydro-1-nitroso-3,5-dinitro-1,3,5-triazine), DNX (hexahydro-1,3-dinitroso-5-nitro-1,3,5-triazine), and TNX (hexahydro-1,3,5-trinitroso-1,3,5-triazine). Under anaerobic conditions, RDX degrades through nitro-reduction, forming the nitroso-derivatives: MNX (hexahydro-1-nitroso-3,5-dinitro-1,3,5-triazine), DNX (hexahydro-1,3-dinitroso-5-nitro-1,3,5-triazine), and TNX (hexahydro-1,3,5-trinitroso-1,3,5-triazine).

RDX can also degrade via ring cleavage pathways leading to: MEDINA (methylenedinitramine), a short-lived intermediate, which further breaks down to formaldehyde and nitrogen gas. Formaldehyde (HCHO) is one of the products after ring cleavage of MEDINA.

RDX F degradation in aerobic conditions results in the formation of NDAB (4-nitro-2,4-diazabutanal).

Another explosive of interest is HMX (1,3,5,7-Tetranitro-1,3,5,7-tetrazocane-C₄H₈N₈O₈), which has a MP of 276-286° C. HMX has the following structure:

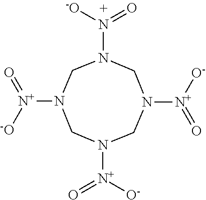

Both of these compounds are expected to produce DNAP and other volatile degradation products, allowing this category of explosives to be identified.

Other explosives can also be tested. These explosives include TNT (trinitrotoluene), ammonium nitrate explosive mixtures, Ammonium perchlorate explosive mixtures, Ammonium picrate, BEAF [1,2-bis-(2, 2-difluoro-2-nitroacetoxyethane)], black powder, BTNEC [bis (trinitroethyl) carbonate], BTNEN [bis (trinitroethyl) nitramine], BTTN [1,2,4-butanetriol trinitrate], calcium nitrate explosive mixture, cellulose hexanitrate explosive mixture, chlorate explosive mixtures, cyclotetramethylenetetranitramine [HMX], DATB [diaminotrinitrobenzene], DDNP [diazodinitrophenol], DEGDN [diethyleneglycol dinitrate], Dimethylol dimethyl methane dinitrate composition, dinitroethyleneurea, dinitroglycerine [glycerol dinitrate], dinitrophenol, dinitrophenolates, dinitrophenyl hydrazine, dinitroresorcinol, dinitrotoluene-sodium nitrate explosive mixtures, DIPAM [dipicramide; diaminohexanitrobiphenyl], dipicryl sulfide [hexanitrodiphenyl sulfide], dipicryl sulfone, dipicrylamine, DNPA [2,2-dinitropropyl acrylate], DNPD [dinitropentano nitrile], EDDN [ethylene diamine dinitrate], EDNA [ethylenedinitramine], ednatol, EDNP [ethyl 4,4-dinitropentanoate], EGDN [ethylene glycol dinitrate], erythritol tetranitrate explosives, esters of nitro-substituted alcohols, ethyl-tetryl, tetranitromethane (nitroform), fulminate of mercury, fulminate of silver, gelatinized nitrocellulose, guanyl nitrosamino guanyl tetrazene, guanyl nitrosamino guanylidene hydrazine, guncotton, hexanitrodiphenylamine, hexanitrostilbene, [hexamethylenetriperoxidediamine], HMX [cyclo-1,3,5,7-tetramethylene, 2,4,6,8-tetranitramine; octogen], KDNBF [potassium dinitrobenzo-furoxane], Lead azide, Lead mannite, lead mononitroresorcinate, lead picrate, mannitol hexanitrate, MDNP [methyl 4,4-dinitropentanoate], MEAN [monoethanolamine nitrate], mercuric fulminate, mercury oxalate, mercury tartrate, metriol trinitrate, MMAN [monomethylamine nitrate]; methylamine nitrate, mononitrotoluene-nitroglycerin mixture, NIBTN [nitroisobutametriol trinitrate], Nitrate sensitized with gelled nitroparaffin, nitrated carbohydrate explosive, nitrated glucoside explosive, nitrated polyhydric alcohol explosives, nitric acid and a nitro aromatic compound explosive, nitric acid and carboxylic fuel explosive, nitric acid explosive mixtures, nitro aromatic explosive mixtures, nitro compounds of furane explosive mixtures, nitrocellulose explosive, nitroderivative of urea explosive mixture, nitrogelatin explosive, nitrogen trichloride, nitrogen tri-iodide, nitroglycerine [NG, RNG, nitro, glyceryl trinitrate, trinitroglycerine], nitroglycide, nitroglycol [ethylene glycol dinitrate, EGDN], nitroguanidine explosives, nitronium perchlorate propellant mixtures, nitroparaffins explosive grade and ammonium nitrate mixtures, nitrostarch, nitro-substituted carboxylic acids, nitrotriazolone [3-nitro-1,2,4-triazol-5-one], nitrourea, silver acetylide, silver azide, silver fulminate, silver oxalate explosive mixtures, silver styphnate, silver tartrate explosive mixtures, silver tetrazene, sodium azide explosive mixture, sodium dinitro-ortho-cresolate, sodium nitrate explosive mixtures, sodium nitrate-potassium nitrate explosive mixture, sodium picramate, tacot [tetranitro-2,3,5,6-dibenzo-1,3a,4,6a-tetrazapentalene], TATB [triaminotrinitrobenzene], TATP [triacetonetriperoxide], TEGDN [triethylene glycol dinitrate], tetranitrocarbazole, tetrazene [tetracene, tetrazine, 1-(5-tetrazolyl)-4-guanyl tetrazene hydrate], tetrazole explosives, tetryl [2,4,6-tetranitro-n-methylaniline], tetrytol, thickened inorganic oxidizer salt slurried explosive mixture, TMETN [trimethylolethane trinitrate], TNEF [trinitroethyl formal], trimethylol ethyl methane trinitrate composition, trimethylolthane trinitrate-nitrocellulose, trimonite, trinitroanisole, trinitrobenzene, trinitrobenzenesulfonic acid [picryl sulfonic acid], trinitrobenzoic acid, trinitrocresol, trinitrofluorenone, trinitro-meta-cresol, trinitronaphthalene, trinitrophenetol, trinitrophloroglucinol, trinitroresorcinol, tritonal and urea nitrate.

The technology of the disclosure has found that using the higher vapor pressures of explosive degradation products as a proxy for explosives odor is an avenue to measuring the presence and magnitude of low vapor pressure explosives. Explosives being high energy materials, tend to naturally exude decomposition products during storage and transport. Moreover, explosives are synthesized, i.e., manufactured in bulk, and there will be the presence of lower molecular weight impurities, accordingly having a higher volatility, that can be measured by sensitive spectroscopy instrumentation. The impurities will also have a distinctive profile that in many cases will identify the particular explosive molecule. The effect can be enhanced by thermally trapping the compounds at a low temperature and then preheating the material to be tested.

Explosives such as RDX can have a high level of impurities. Commercially manufactured RDX is an impurity level of approximately 10% HMX. Other impurities in RDX include urotropin (hexamethylenetetramine—(CH₂)₆N₄), acetic anhydride, acetic acid and triazine. RDX biodegradation can occur both in aerobic and anaerobic environments during its storage. Heating RDX at 70° C. exudes cyclohexanone. Impurities arising from the synthesis of RDX and HMX (Bachman Process) include the raw materials from manufacture of these products, including hexamine, ammonium nitrate, nitric acid, and acetic acid. RDX also appears as an impurity in HMX.

Vapor pressure, i.e., partial vapor pressure, is calculated using the Clausius-Clapeyron equation:

$$ln {P}_{vap} = ( \frac{- Δ {H}_{vap}}{R} ) \frac{1}{T} + C$$

where Pᵥₐₚ is the vapor pressure, ΔHᵥₐₚ is the heat of vaporization, R is the gas constant, T is temperature and C is a constant of the specific liquid. The vapor pressure of some typical explosives can be seen in Table 1.

TABLE 1
Vapor Pressure of explosives.

| Explosive | Vapor Pressure | Temp. (ºC.) |
|---|---|---|
| RDX | 3 ± 1 x 10⁻¹¹ atm (30 ppt) | 25 |
| HMX | 1.0 ± 0.6 x 10⁻¹⁵ atm (1.0 ppq) | 25 |
| TNT | 2.63 x 10⁻⁷ atm (0.0002 mm Hg) | 20 |
| Nitroglycerin | 2.42 x 10⁻⁷ atm (2.6 x 10⁻⁴ mm Hg) | 20 |
| TATP | 6.6 x 10⁻⁴ atm (0.5 mm Hg) | 20 |
| Sodium nitrate | (7.44 x 10⁻¹⁷ mm Hg) | 20 |

As can be seen, there is a wide range of vapor pressure that can go down to ppq (parts per quadrillion) levels or lower for the less volatile molecules.

The technology of the disclosure can even detect RDX and HMX at ppb levels when their vapor pressures are 10,000 and 1,000,000 times lower. The degradation products are the source of the signal. Sodium Nitrate and or potassium nitrate as used in black powder has also been detected.

Among the explosive detection methods of the disclosure is Thermal Decomposition Cavity Ring-Down Spectroscopy (TD-CRDS). TD-CRDS is a form of laser absorption spectroscopy that utilizes a thermal decomposition cavity in a uv-visible spectrometer that utilizes screen mirrors, lasers, filters and a sample cell. A typical CRDS setup is formed from a laser that is used to illuminate a high-finesse optical cavity, which in its simplest form consists of two or three highly reflective mirrors. When the laser is in resonance with a cavity mode, intensity builds up in the cavity due to the multiple reflections. The laser is then turned off in order to allow the measurement of the exponentially decaying light intensity leaking from the cavity. During this decay, light is reflected back and forth thousands of times between the mirrors giving an effective path length for the extinction on the order of a kilometer. When the light is turned back on, the buildup of light can also be measured as an indication of the time constant.

If a light-absorbing material is now placed in the cavity, the mean lifetime decreases as fewer bounces through the medium are required before the light is fully absorbed, or absorbed to some fraction of its initial intensity. A TD-CRDS setup measures how long it takes for the light to decay to 1/e of its initial intensity, and this “ringdown time” can be used to calculate the concentration of the absorbing substance in the gas mixture in the cavity. There may be extra steps to reduce the noise to close to the photon limit.

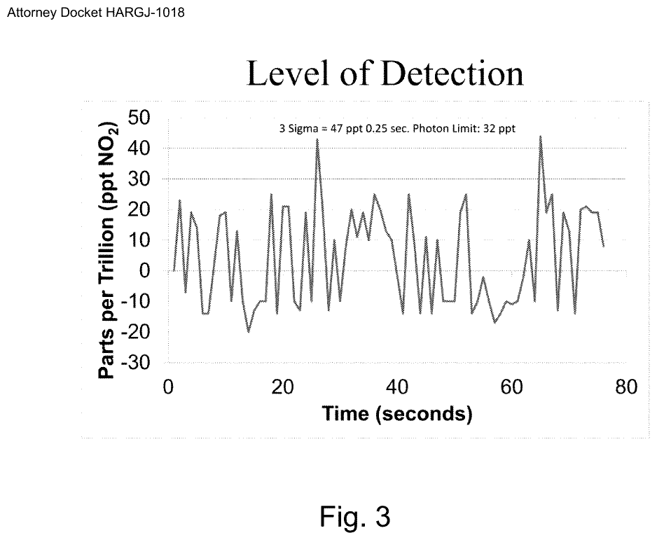

A CRDS detector is at least a 1000 times more sensitive than a flame ionization detector (FID). FIG. 3 shows the threshold level for TNT for a CRDS measurement of a sample according to an embodiment of the disclosure. As can be seen, TNT is detected close to the photon limit with a sensitivity of 9 parts per trillion (PPT) at a path length of 937 m. Further adjustments to the CRDS detector can bring the sensitivity down to a 1 PPT level or lower. At these levels, there is no need to open the vial or the package containing the vial.

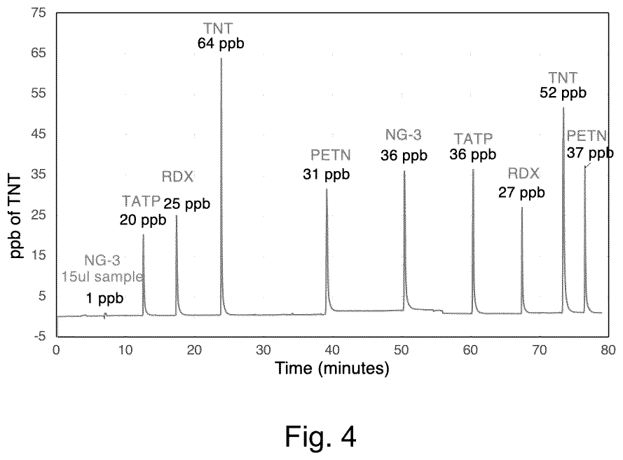

This high level of sensitivity as achievable for a wide range of explosive materials, as can be seen in FIG. 4. A high level of sensitivity with residence time was observed for materials that included nitroglycerin (NG), hexahydro-1,3,5-trinitro-s-triazine (RDX), 2,4,6-trinitotoluene (TNT), triacetone triperoxide (TATP), and pentaerythritol tetranitrate (PETN). For each of these compounds, linearity measurements were taken, plotting the analyzers' responses to variable concentrations. Although many of the peaks are at 5 ppb or higher, the bottom of the peaks indicate that sensitivity down the PPT level is possible. A 15 μl sample of NG displayed a sensitivity of 1 PPB. Thermal decomposition can also have a peroxy radical chain amplification effect so that multiple NO₂ molecules come from each generated radical with the addition of NO and the presence of atmospheric oxygen.

Other detection methods besides CRDS may be utilized. Testing of small samples of explosives includes complicated analytical systems, for examples a mass spectrometer. The mass spectrometer fulfils the main performance requirements of an explosive detection system, which includes sensitivity, selectivity, and speed of analysis. In addition, there are requirements of mobility and cost as well. The mass spectrometers have become a lot smaller, mobile, and less expensive during recent years. Various mass spectrometer configurations have been used, which includes ion traps, quadrupoles, and Time-of-Flight (ToF) analyzers and tandem mass spectrometers (MS/MS) combinations for the purpose of detection of explosives.

Mass spectrometry deals with the separation and analysis of substances according to the masses of atoms and molecules of which the substance is composed. There are two basic methods of separation and analysis, which include methods based on time separation and the methods based on geometric separation. The time separation method is based on the fact that ions having different m/e ratios have different times of flight and are thus collected one after the other. While on the other hand, in the geometric separation method, ions having different m/e ratios are separated according to their geometric position at the collecting spot. Because of its high sensitivity and selectivity, the mass spectrometry has always shown great potential for vapor and trace detection of explosives. Due to the necessity of transferring the sample from atmospheric levels to low vacuum levels needed for the mass spectroscopy, the equipment can be difficult to keep working and maintain.

Other methods to detect and characterize explosives include visual detection fluorescence methods that are simple, have fast detection times as well as the possibility of the fine structural tuning of chemosensor/sensory material to improve the selectivity and/or desired photophysical properties. Other physical methods can include chromatographic methods, high-performance liquid chromatography, (surface-enhanced) Raman spectroscopy, terahertz absorption spectroscopy, photoacoustic spectroscopy, immunoassay electrochemical methods. Fluorescence-chemosensors based methods are also considered to be viable. Other methods include detection of traces of explosives by means of Fourier Transform Infrared Spectroscopy (FTIR).

The technology of the disclosure is not restricted to explosives. Any compound that has even a trace volatility can be detected and quantified. The PPT sensitivity renders this technology for the detection of narcotics such as morphine, heroin and fentanyl, hallucinogens including LSD, PCP, mescaline, MDA and psilocybin, and illegal pharmaceuticals such as steroids. Also, the animal training protocols include exposing the animal to interfering scents such as vanilla, peanut butter, rose, mint, clove, pepper, etc., Animal scents used in dog training include bear, duck, dove, fox, goose, grouse, pheasant, quail, rabbit, raccoon, squirrel, and wild boar.

While various aspects have been described above, it should be understood that they have been presented by way of example only, and not limitation. Thus, the breadth and scope of an aspect of the present invention should not be limited by any of the above-described exemplary aspects, but should be defined only in accordance with the following claims and their equivalents.

What is claimed is:

1. A method of sensing explosives, comprising:
sampling an atmosphere over a sample; and
detecting explosive impurities or decomposition products in the atmosphere.

2. The method of claim 1, wherein the detecting is performed by thermal decomposition cavity ring-down spectroscopy, or by thermal desorption thermal decomposition cavity ring-down spectroscopy.

3. The method of claim 1, wherein the detecting is performed by thermal decomposition laser induced fluorescence, or by thermal desorption thermal decomposition laser induced fluorescence.

4. The method of claim 1, wherein the explosives are at least one of cyclotrimethylene trinitramine, 1,3,5,7-Tetranitro-1,3,5,7-tetrazocane, TNT, PETN, nitroglycerin, triaminotrinitrobenzene, triacetonetriperoxide, potassium nitrate or sodium nitrate.

5. The method of claim 1, wherein the explosive is selected from the group consisting of cyclotrimethylene trinitramine, ammonium perchlorate, ammonium picrate, 1,2-bis-(2,2-difluoro-2-nitroacetoxyethane), black powder, bis (trinitroethyl) carbonate, bis (trinitroethyl) nitramine, 1,2,4-butanetriol trinitrate, calcium nitrate explosive mixture, cellulose hexanitrate explosive mixture, chlorate explosive mixtures, cyclotetramethylenetetranitramine, diaminotrinitrobenzene, diazodinitrophenol, diethyleneglycol dinitrate, dimethylol, dinitroethyleneurea, dinitroglycerine, dinitrophenol, dinitrophenolates, dinitrophenyl hydrazine, dinitroresorcinol, dinitrotoluene-sodium nitrate explosive mixtures, dipicramide; diaminohexanitrobiphenyl, dipicryl sulfide, dipicryl sulfone, dipicrylamine, 2,2-dinitropropyl acrylate, dinitropentano nitrile, ethylene diamine dinitrate, ethylenedinitramine, ednatol, ethyl 4,4-dinitropentanoate, ethylene glycol dinitrate, erythritol tetranitrate explosives, esters of nitro-substituted alcohols, ethyl-tetryl, tetranitromethane, fulminate of mercury, fulminate of silver, gelatinized nitrocellulose, guanyl nitrosamino guanyl tetrazene, guanyl nitrosamino guanylidene hydrazine, guncotton, hexanitrodiphenylamine, hexanitrostilbene, hexamethylenetriperoxidediamine, cyclo-1,3,5,7-tetramethylene, 2,4,6,8-tetranitramine; octogen, potassium dinitrobenzo-furoxane, lead azide, lead mannite, lead mononitroresorcinate, lead picrate, mannitol hexanitrate, methyl 4,4-dinitropentanoate, monoethanolamine nitrate, mercuric fulminate, mercury oxalate, mercury tartrate, metriol trinitrate, monomethylamine nitrate, methylamine nitrate, mononitrotoluene-nitroglycerin mixture, nitroisobutametriol trinitrate, nitrate sensitized with gelled nitroparaffin, nitrated carbohydrate explosive, nitrated glucoside explosive, nitrated polyhydric alcohol explosives, nitric acid and a nitro aromatic compound explosive, nitric acid and carboxylic fuel explosive, nitric acid explosive mixtures, nitro aromatic explosive mixtures, nitro compounds of furane explosive mixtures, nitrocellulose explosive, nitroderivative of urea explosive mixture, nitrogelatin explosive, nitrogen trichloride, nitrogen tri-iodide, nitroglycerine, nitroglycide, nitroglycol, nitroguanidine explosives, nitronium perchlorate propellant mixtures, nitroparaffins explosive grade and ammonium nitrate mixtures, nitrostarch, nitro-substituted carboxylic acids, 3-nitro-1,2,4-triazol-5-one, nitrourea, silver acetylide, silver azide, silver fulminate, silver oxalate explosive mixtures, silver styphnate, silver tartrate explosive mixtures, silver tetrazene, sodium azide explosive mixture, sodium dinitro-ortho-cresolate, sodium nitrate explosive mixtures, sodium nitrate-potassium nitrate explosive mixture, sodium picramate, tetranitro-2,3,5,6-dibenzo-1,3a,4,6a-tetrazapentalene, triaminotrinitrobenzene, triacetonetriperoxide, triethylene glycol dinitrate, tetranitrocarbazole, tetrazene tetracene, tetrazine, 1 (5-tetrazolyl)-4-guanyl tetrazene hydrate, tetrazole explosives, tetryl 2,4,6-tetranitro-n-methylaniline, tetrytol, thickened inorganic oxidizer salt slurried explosive mixture, trimethylolethane trinitrate, trinitroethyl formal, trimethylol ethyl methane trinitrate composition, trimethylolthane trinitrate-nitrocellulose, trimonite, trinitroanisole, trinitrobenzene, trinitrobenzenesulfonic acid picryl sulfonic acid, trinitrobenzoic acid, trinitrocresol, trinitrofluorenone, trinitro-meta-cresol, trinitronaphthalene, trinitrophenetol, trinitrophloroglucinol, trinitroresorcinol, tritonal and urea nitrate.

6. The method of claim 1, wherein the detecting is at parts per billion and parts per trillion levels.

7. The method of claim 1, wherein cyclotrimethylene trinitramine and 1,3,5,7-Tetranitro-1,3,5,7-tetrazocane are detected at parts per billion or parts per trillion levels when their vapor partial pressures are 10,000 and 1,000,000 times lower.

8. An apparatus that performs the method of claim 1, comprising:
an inlet;
a particulate filter; and
a detector.

9. The apparatus of claim 8, further comprising a thermoelectric element configured to heat or cool the sample and an optional capillary column before the inlet.

10. The apparatus of claim 8, wherein there is a NO generator configured to feed NO into the atmosphere prior to the detector.

11. The apparatus of claim 8, wherein the detector is a thermal decomposition cavity ring-down spectrometer, or by a thermal desorption thermal decomposition cavity ring-down spectrometer.

12. The apparatus of claim 8, wherein the detector is a thermal decomposition laser induced fluorescence detector, or a thermal desorption thermal decomposition laser induced fluorescence detector.

13. A method of sensing explosives, comprising:
sampling an atmosphere over a sample; and
detecting explosive impurities or decomposition products in the atmosphere, wherein the explosives include at least one of cyclotrimethylene trinitramine, 1,3,5,7-Tetranitro-1,3,5,7-tetrazocane, TNT, PETN, nitroglycerin, triaminotrinitrobenzene, triacetonetriperoxide or sodium nitrate.

14. The method of claim 13, wherein the detecting is performed by thermal decomposition cavity ring-down spectroscopy, or by thermal desorption thermal decomposition cavity ring-down spectroscopy.

15. The method of claim 13, wherein the detecting is performed by thermal decomposition laser induced fluorescence, or by thermal desorption thermal decomposition laser induced fluorescence.

16. The method of claim 13, wherein cyclotrimethylene trinitramine and 1,3,5,7-Tetranitro-1,3,5,7-tetrazocane are detected at parts per billion levels when their vapor pressures are 10,000 and 1,000,000 times lower.

17. An apparatus that performs the method of claim 13, comprising:
an inlet;
a particulate filter; and
a detector.

18. The apparatus of claim 17, further comprising a thermoelectric element configured to heat or cool the sample and an optional capillary column before the inlet.

19. The apparatus of claim 17, wherein there is an NO generator configured to feed NO into the atmosphere prior to the detector.

20. The apparatus of claim 17, wherein the detector is a thermal decomposition cavity ring-down spectrometer, a thermal desorption thermal decomposition cavity ring-down spectrometer, a thermal decomposition laser induced fluorescence detector, or a thermal desorption thermal decomposition laser induced fluorescence detector.
